(19) United States
(12) Patent Application Publication
(10) Pub. No.: US 2025/0276133 A1
(43) Pub. Date: Sep. 4, 2025

(54) ATOMIZER

(71) Applicant: Suzhou Boyunxing Technology Co., Ltd., Suzhou (CN)

(72) Inventor: Feng WANG, Suzhou (CN)

(73) Assignee: Suzhou Boyunxing Technology Co., Ltd., Suzhou (CN)

(21) Appl. No.: 19/210,821

(22) Filed: May 16, 2025

Related U.S. Application Data
(63) Continuation of application No. PCT/CN2023/132423, filed on Nov. 17, 2023.

(30) Foreign Application Priority Data
Nov. 24, 2022 (CN) 202211485092.8

(51) Int. Cl. A61M 11/00

(52) U.S. Cl. CPC A61M 11/001; A61M 2205/00

(57) ABSTRACT

An atomizer is provided. The atomizer includes: a mouthpiece, a first end of which being configured to be placed at a user's mouth; a delivery tube holder located at a second end of the mouthpiece opposite to the first end; a guide mechanism for guiding the delivery tube holder to move between a first position and a third position in an axial direction of the mouthpiece, the delivery tube holder is closest to the mouthpiece in the axial direction when it is in the third position, and the delivery tube holder is farthest from the mouthpiece in the axial direction when it is in the first position; the atomizer performs spraying during the movement of the delivery tube holder from the first position to the third position, and the atomizer performs liquid metering during the movement of the delivery tube holder from the third position to the first position.

### CROSS-REFERENCE TO RELATED APPLICATIONS

The present application is a continuation of the international application PCT/CN2023/132423, filed on Nov. 17, 2023 and entitled “ATOMIZER”, and the international application claims the priority to Chinese Patent Application No. 202211485092.8 filed on Nov. 24, 2022, which is incorporated herein by reference in its entirety.

### TECHNICAL FIELD

The disclosure relates to the technical field of medical instruments. Specifically, the disclosure relates to an atomizer which may be used, for example, for atomizing and/or spraying a medicinal liquid.

### BACKGROUND

In the related art, a container in an atomizer (or referred to as a sprayer) contains a liquid to be atomized or sprayed, and during a movement stroke of the container relative to a spraying assembly (such as a pump), the liquid in the container is atomized, and the atomized liquid is sprayed from a nozzle of the spraying assembly.

### SUMMARY OF THE INVENTION

The disclosure provides an atomizer.

According to one aspect of the disclosure, an atomizer is provided. The atomizer includes: a mouthpiece, a first end of the mouthpiece being configured to be placed at a mouth of a user; a delivery tube holder located at a second end of the mouthpiece opposite to the first end; and a guide mechanism configured to guide the delivery tube holder to move between a first position and a third position in an axial direction of the mouthpiece, where the delivery tube holder is closest to the mouthpiece in the axial direction when the delivery tube holder is in the third position, and the delivery tube holder is farthest from the mouthpiece in the axial direction when the delivery tube holder is in the first position; and where the atomizer performs spraying during the movement of the delivery tube holder from the first position to the third position, and the atomizer performs liquid metering during the movement of the delivery tube holder from the third position to the first position.

According to some embodiments, there is a second position between the first position and the third position, the guide mechanism is configured to guide the delivery tube holder to move between the first position, the second position and the third position in the axial direction of the mouthpiece, the atomizer performs a first spraying operation during the movement of the delivery tube holder from the first position to the second position, and the atomizer performs a second spraying operation during the movement of the delivery tube holder from the second position to the third position.

According to some embodiments, the guide mechanism includes: a first guide structure arranged on an inner wall of the delivery tube holder, with a surface of the first guide structure facing the mouthpiece forming a first guide surface; and a second guide structure arranged on an inner wall of the mouthpiece, with a surface of the second guide structure facing the delivery tube holder forming a second guide surface.

According to some embodiments, when a first portion of the first guide surface abuts on a second portion of the second guide surface, the delivery tube holder is in the first position; when a third portion of the first guide surface abuts on a fourth portion of the second guide surface, the delivery tube holder is in the second position; and when the first guide surface is separated from the second guide surface, the delivery tube holder is in the third position.

According to some embodiments, the first guide surface includes: a guide portion configured to guide the delivery tube holder to move from the third position to the first position; and a stop portion configured to guide the delivery tube holder to move from the first position to the second position.

According to some embodiments, the guide portion is an inclined surface, and the stop portion is a step surface and includes an axial flat portion parallel to the axial direction and a radial flat portion parallel to a radial direction of the delivery tube holder.

According to some embodiments, the first portion is the highest point of the guide portion in the axial direction, and the second portion is the highest point of the second guide surface in the axial direction; and the third portion is the radial flat portion of the stop portion, and the fourth portion is the same portion as the second portion.

According to some embodiments, the first guide surface further includes a transition portion located between the guide portion and the stop portion and configured as a cambered surface.

According to some embodiments, the width of the first guide surface in a radial direction of the delivery tube holder is different from the width of the second guide surface in the radial direction.

According to some embodiments, the first guide structure is integrally formed with the delivery tube holder, and the second guide structure is integrally formed with the mouthpiece.

According to some embodiments, the atomizer further includes an actuating member, where the actuating member is capable of being actuated to drive the delivery tube holder to pivot relative to the mouthpiece, and the pivoted delivery tube holder is capable of being guided by the guide mechanism to move in the axial direction.

According to some embodiments, the atomizer further includes a locking mechanism, where the locking mechanism switches the actuating member between a locked position, in which the mouthpiece stops the actuating member from pivoting, and an unlocked position, in which the actuating member is capable of pivoting relative to the mouthpiece.

According to some embodiments, when the delivery tube holder is in the first position and the third position, the actuating member is in the unlocked position; and when the delivery tube holder is in the second position, the actuating member is in the locked position.

According to some embodiments, the atomizer further includes a switch button, where in the locked position, the switch bottom is capable of being pressed to drive the delivery tube holder to pivot to separate the first guide surface from the second guide surface.

According to some embodiments, the atomizer further includes a bottom spring arranged at the bottom of the delivery tube holder, where the bottom spring is configured to apply a thrust to the delivery tube holder to drive the delivery tube holder to move toward the mouthpiece, and when the first guide surface is separated from the second guide surface, the delivery tube holder is capable of moving from the second position to the third position under the action of the thrust of the bottom spring.

According to some embodiments, the locking mechanism includes: a locking slider provided on the mouthpiece; a slot provided in a circumferential wall of the actuating member; and a protrusion provided on a circumferential surface of the delivery tube holder and extending into the slot, where when the delivery tube holder is in the first position, the locking slider abuts against an end surface of the actuating member facing the mouthpiece, such that the actuating member is in the unlocked position; where when the delivery tube holder is in the second position, a portion of the locking slider is inserted into the slot and abuts on the protrusion, such that the actuating member is in the locked position; and where when the delivery tube holder is in the third position, the locking slider is pushed by the protrusion to return to the mouthpiece, such that the actuating member is in the unlocked position.

According to some embodiments, when the delivery tube holder is in the first position, the atomizer is in a pre-cleaning state; when the delivery tube holder is in the second position, the atomizer is in a cleaned state and a pre-triggering state; and when the delivery tube holder is in the third position, the atomizer is in a triggered state or an initial state.

According to some embodiments, when the delivery tube holder is in the first position, the actuating member is in the locked position; and when the delivery tube holder is in the third position, the actuating member is in the unlocked position.

According to some embodiments, the locking mechanism includes: a locking slider provided on the mouthpiece; a slot provided in a circumferential wall of the actuating member; and a protrusion provided on a circumferential surface of the delivery tube holder and extending into the slot, where when the delivery tube holder is in the first position, a portion of the locking slider is inserted into the slot and abuts on the protrusion, such that the actuating member is in the locked position; and where when the delivery tube holder is in the third position, the locking slider is pushed by the protrusion to return to the mouthpiece, such that the actuating member is in the unlocked position.

### BRIEF DESCRIPTION OF THE DRAWINGS

To describe technical solutions in embodiments of the disclosure or in the prior art more clearly, accompanying drawings required for description of the embodiments or the prior art will be briefly introduced below. Apparently, the accompanying drawings in the following description show some embodiments of the disclosure merely, and those of ordinary skill in the art can derive other drawings from structures shown in these accompanying drawings without any creative effort.

In the figures:

FIG. 1 shows a front view of an atomizer according to some embodiments of the disclosure;

FIG. 2 shows a cross-sectional view of the atomizer in FIG. 1 taken along R-R;

FIG. 3 shows an exploded view of the atomizer in FIG. 1;

FIGS. 4A to 4D show schematic diagrams of the atomizer in FIG. 1 in a pre-cleaning state from different perspectives, FIG. 4A showing a cross-sectional view of the atomizer in FIG. 1 in the pre-cleaning state taken along N-N, FIG. 4B showing a schematic diagram of some components of the atomizer in the pre-cleaning state, FIG. 4C showing a cross-sectional view of some components in FIG. 4B taken along K-K, and FIG. 4D showing an enlarged view of an area H of the atomizer in FIG. 4A;

FIGS. 5A to 5D show schematic diagrams of the atomizer in FIG. 1 in a cleaned state from different perspectives, FIG. 5A showing a cross-sectional view of the atomizer in FIG. 1 in the cleaned state taken along N-N, FIG. 5B showing a schematic diagram of some components of the atomizer in the cleaned state, FIG. 5C showing a cross-sectional view of some components in FIG. 5B taken along D-D, and FIG. 5D showing an enlarged view of an area P of the atomizer in FIG. 5A;

FIGS. 6A to 6D show schematic diagrams of the atomizer in FIG. 1 in a triggered state from different perspectives, FIG. 6A showing a cross-sectional view of the atomizer in FIG. 1 in the triggered state taken along N-N, FIG. 6B showing a schematic diagram of some components of the atomizer in the triggered state, FIG. 6C showing a cross-sectional view of some components in FIG. 6B taken along E-E, and FIG. 6D showing an enlarged view of an area Q of the atomizer in FIG. 6A;

FIG. 7A shows a schematic diagram of a delivery tube holder of the atomizer in FIG. 1;

FIG. 7B shows a top view of the delivery tube holder in FIG. 7A;

FIG. 8A shows a schematic diagram of a mouthpiece of the atomizer in FIG. 1;

FIG. 8B shows a top view of the mouthpiece in FIG. 8A;

FIG. 9 shows a schematic diagram of a locking slider of the atomizer in FIG. 1;

FIG. 10 shows a schematic diagram of an actuating member of the atomizer in FIG. 1;

FIG. 11 shows a schematic diagram of a switch button of the atomizer in FIG. 1; and

FIGS. 12A to 12C show schematic diagrams of an atomizer according to some other embodiments of the disclosure in a pre-triggering state from different perspectives, FIG. 12A showing a cross-sectional view of the atomizer in the pre-triggering state taken along a vertical direction, FIG. 12B showing a schematic diagram of some components of the atomizer in the pre-triggering state, and FIG. 12C showing a cross-sectional view of some components in FIG. 12B taken along D′-D′; and

FIGS. 13A to 13C show schematic diagrams of the atomizer in FIG. 12A in a triggered state from different perspectives, FIG. 13A showing a cross-sectional view of the atomizer in the triggered state taken along a vertical direction, FIG. 13B showing a schematic diagram of some components of the atomizer in the triggered state, and FIG. 13C showing a cross-sectional view of some components in FIG. 13B taken along E′-E′.

In the figures, the same reference signs refer to the same or similar features.

The implementation of the objective, functional characteristics, and advantages of the disclosure will be further described with reference to the accompanying drawings in combination with embodiments.

### DETAILED DESCRIPTION

In the scope of the disclosure, an “atomizer” (also referred to as a “sprayer”) refers to a device for atomizing a liquid. Generally, the atomizer is configured to atomize a fluid (e.g., a medicinal liquid or the like) and spray the atomized fluid to some parts of a user (such as a patient) to be treated. Since the medicinal liquid is loaded in the atomizer, the stability of the atomizer is particularly important.

In the related art, the atomizer is usually susceptible to contaminants in the environment, residual medicinal liquid from the previous spraying, and a medicinal liquid volatilized into a spraying assembly before formal spraying, resulting in unstable drug delivery dose from the atomizer and thus affecting the efficacy of the medicinal liquid.

In the disclosure, a delivery tube holder is guided by a guide mechanism to move between a first position, a second position and a third position, so as to perform two spraying operations, that is, a first spraying operation (pre-spraying) and a second spraying operation (formal drug-delivery spraying) within one stroke cycle. The pre-spraying can prevent the residual medicinal liquid in the spraying assembly from mixing in the formal drug-delivery spraying and thus stabilize the dose of the medicinal liquid, and can also remove contaminants at the nozzle of a nozzle assembly to prevent the contaminants from being inhaled with the drug and thus achieve a cleaning effect. This allows for safer, more stable, more reliable and more effective drug delivery.

In the scope of the disclosure, the “locked position” of the actuating member refers to a position in which the actuating member is locked, that is, the atomizer cannot be triggered without the action of other components. In other words, when the actuating member is in the “locked position”, the atomizer can perform an atomization or spraying operation only manually by means of other components.

In the scope of the disclosure, the “unlocked position” of the actuating member refers to a position in which the actuating member is not locked, that is, a position in which the atomizer can be operated by means of the actuating member. In other words, when the actuating member is in the “unlocked position”, a user can rotate the actuating member to drive the delivery tube holder to perform corresponding rotation and axial movement at the same time.

In the scope of the disclosure, the “initial state” of the atomizer refers to a state in which no operation is performed on the atomizer, the “pre-cleaning state” refers to a critical state of the atomizer before pre-spraying, the “cleaned state” refers to a state in which the atomizer has completed the pre-spraying, that is, a pre-spraying operation has just been completed and no other operations are performed, the “pre-triggering state” refers to a critical state of the atomizer before formal drug-delivery spraying, and the “triggered state” refers to a state in which the atomizer has completed the formal drug-delivery spraying, that is the spraying has just been completed and no other operations are performed. In some examples, the “cleaned state” and the “pre-triggering state” are the same state, specifically, the components in the atomizer are in the same positions and states.

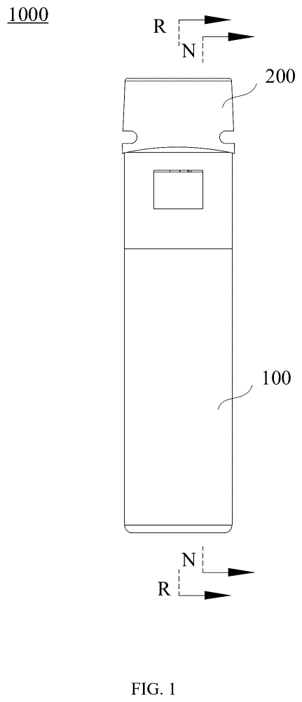

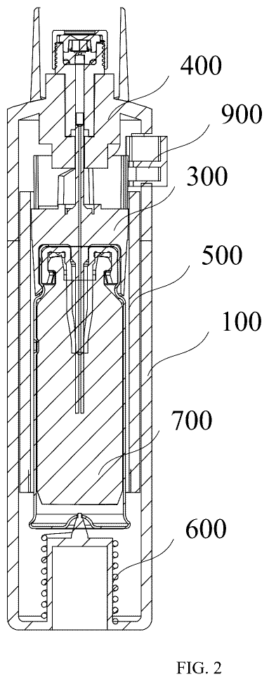

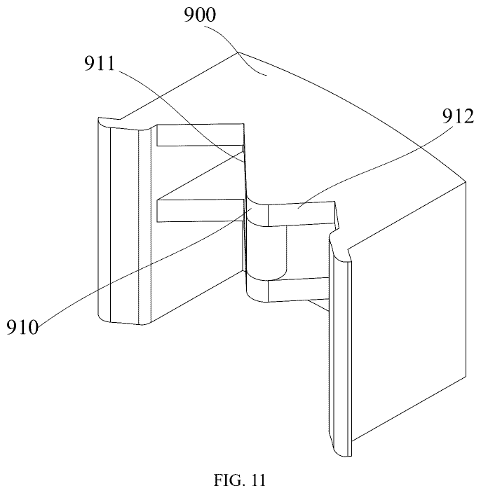

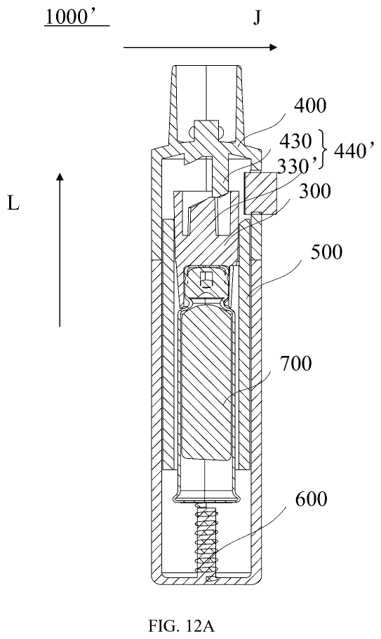

An atomizer according to some specific embodiments of the disclosure will be described in detail below with reference to the embodiments shown in FIGS. 1 to 13.

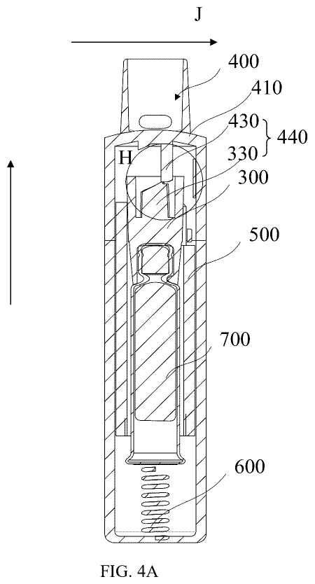

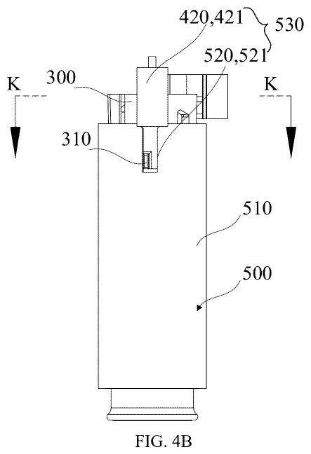

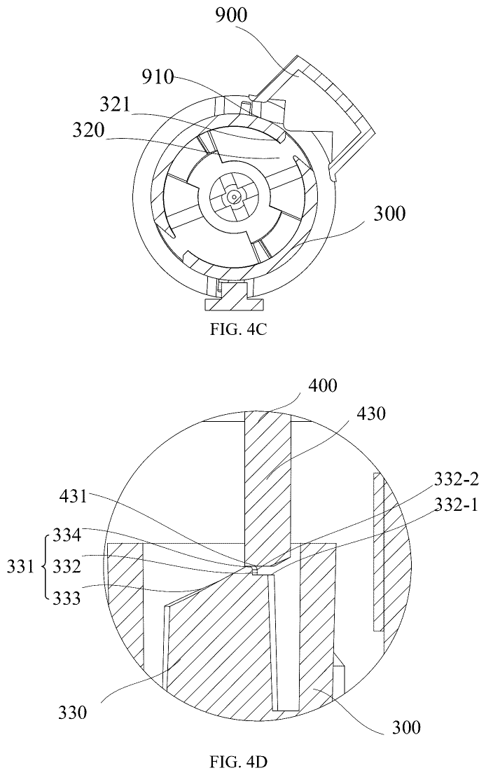

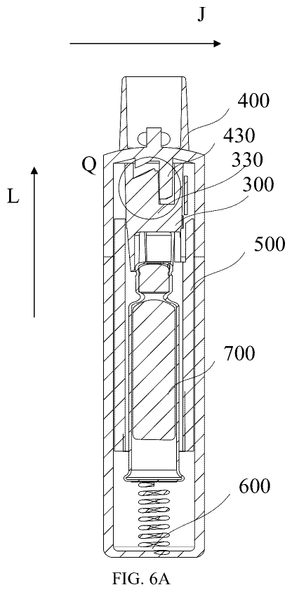

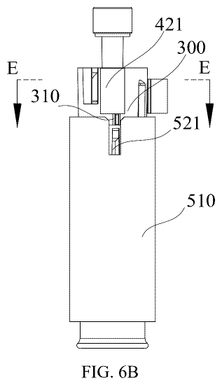

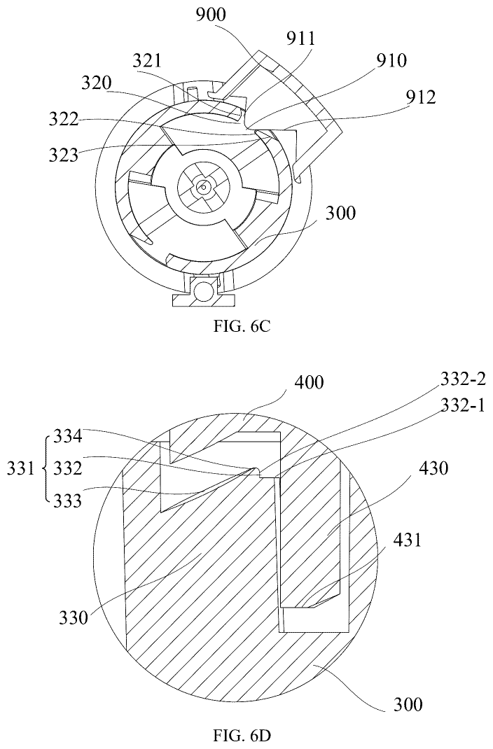

As shown in FIGS. 1, 2 and 3, an atomizer 1000 may include a mouthpiece 400, a delivery tube holder 300 and a guide mechanism 440. The guide mechanism 440 is configured to guide the delivery tube holder 300 to move between a first position (as shown in FIG. 4A) and a third position (as shown in FIG. 6A) in an axial direction L of the mouthpiece 400. The delivery tube holder 300 is closest to the mouthpiece 400 in the axial direction L when the delivery tube holder is in the third position, and the delivery tube holder 300 is farthest from the mouthpiece 400 in the axial direction L when the delivery tube holder is in the first position. The atomizer 1000 performs spraying during the movement of the delivery tube holder 300 from the first position to the third position, and the atomizer 1000 performs liquid metering during the movement of the delivery tube holder 300 from the third position to the first position. Liquid metering herein refers to a liquid being drawn into a metering chamber of a spraying assembly 800, such that the liquid (i.e., a medicinal liquid) is ready before the spraying.

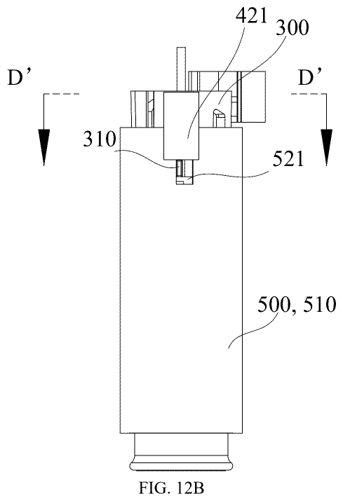

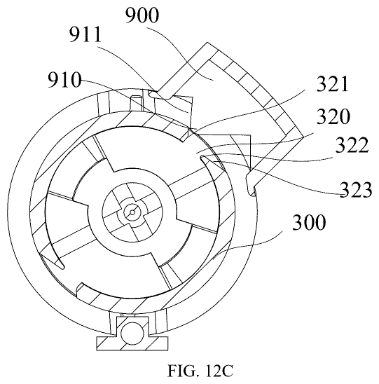

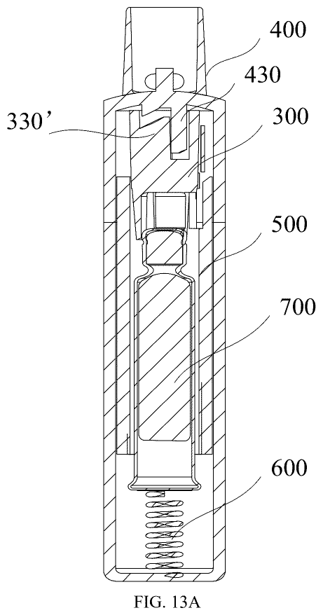

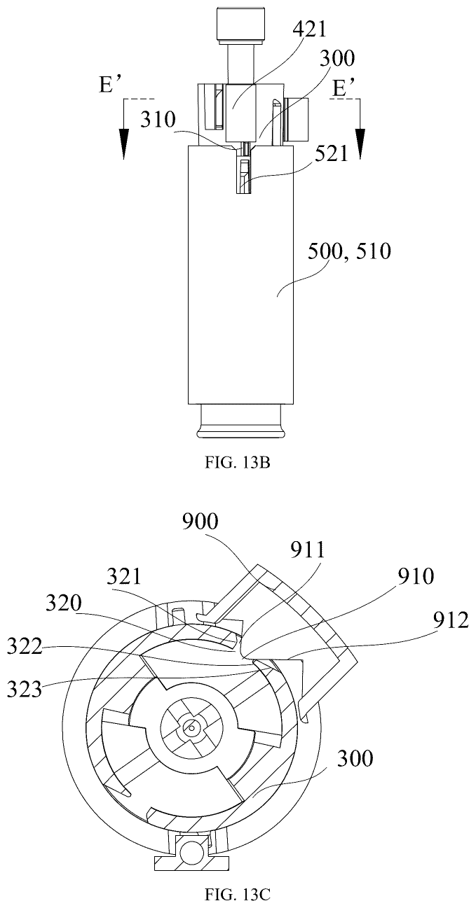

In some embodiments, as shown in FIGS. 12A to 13C, the guide mechanism 440′ may be configured to guide the delivery tube holder 300 to move between the first position (as shown in FIG. 12A) and the third position (as shown in FIG. 13A) in the axial direction L of the mouthpiece 400, so as to achieve spraying of the atomizer. That is, as shown in FIG. 12A, when the delivery tube holder 300 is in the first position, the delivery tube holder 300 is in the lowest position in the axial direction L. As shown in FIG. 13A, when the delivery tube holder 300 is in the third position, the delivery tube holder 300 is in the highest position in the axial direction L, that is, the delivery tube holder moves a distance upward from the first position in the axial direction. Thus, during the movement stroke of the delivery tube holder 300 from the first position to the third position, the liquid in a container 700 is atomized and spayed from a nozzle of the spraying assembly 800, such that a spraying operation (i.e., the formal drug-delivery spraying) is performed. During the movement from the third position to the first position, the liquid in the container 700 is dawn into the metering chamber of the spraying assembly 800 and can thus be reused.

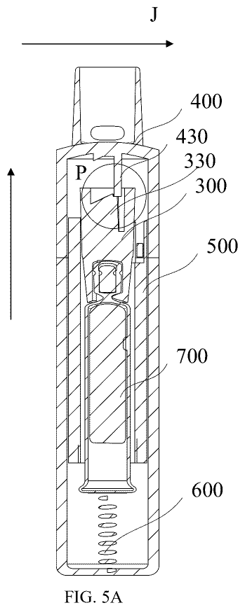

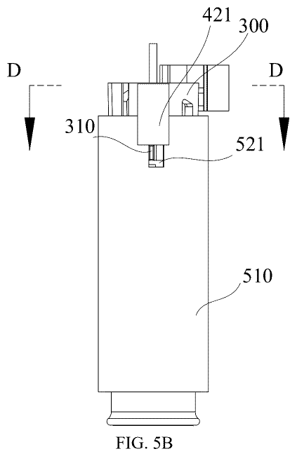

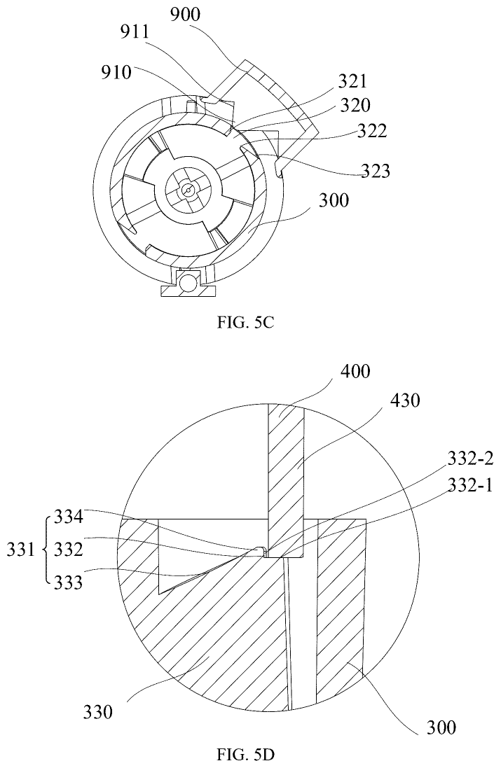

In some embodiments, as shown in FIGS. 4A to 6D, there is a second position between the first position and the third position. In this case, the guide mechanism 440 may be configured to guide the delivery tube holder 300 to move between the first position (as shown in FIG. 4A), the second position (as shown in FIG. 5A) and the third position (as shown in FIG. 6A) in the axial direction L of the mouthpiece 400. The atomizer 1000 performs liquid metering during the movement of the delivery tube holder 300 from the third position to the first position, the atomizer 1000 performs a first spraying operation during the movement of the delivery tube holder 300 from the first position to the second position, and the atomizer 1000 performs a second spraying operation during the movement of the delivery tube holder 300 from the second position to the third position, so as to complete one dosing cycle.

That is, as shown in FIG. 4A, when the delivery tube holder 300 is in the first position, the delivery tube holder 300 is in the lowest position in the axial direction L. As shown in FIG. 5A, when the delivery tube holder 300 is in the second position, the delivery tube holder 300 is in an intermediate position between the highest position and the lowest position in the axial direction L, that is, the delivery tube holder moves a distance upward from the first position in the axial direction. As shown in FIG. 6A, when the delivery tube holder 300 is in the third position, the delivery tube holder 300 is in the highest position in the axial direction L, that is, the delivery tube holder moves a distance upward from the second position in the axial direction. Thus, during a movement stroke of the delivery tube holder 300 from the first position to the second position, the liquid in a container 700 connected to the delivery tube holder 300 is atomized and spayed from a nozzle of the spraying assembly 800, such that the first spraying operation (i.e., the pre-spraying) is performed. During a movement stroke of the delivery tube holder 300 from the second position to the third position, the liquid in the container 700 is atomized and spayed from the nozzle of the spraying assembly 800, such that the second spraying operation (i.e., the formal drug-delivery spraying) is performed. During the movement from the third position to the first position, the liquid in the container 700 is dawn into the metering chamber of the spraying assembly 800 and can thus be reused. The atomizer 1000 is guided by the guide mechanism 440 to move between the first position, the second position and the third position, so as to perform two spraying operations, that is, the pre-spraying and the formal spraying, within one stroke cycle. The pre-spraying before the formal spraying can prevent the residual medicinal liquid in the spraying assembly 800 from mixing in the formal drug-delivery spraying and thus stabilize the dose of the medicinal liquid, and can also remove contaminants at the nozzle of a nozzle assembly to prevent the contaminants from being inhaled with the drug and thus achieve a cleaning effect. This allows for safer, more stable, more reliable and more effective drug delivery.

An atomizer 1000 according to some specific embodiments of the disclosure in which pre-spraying is performed will be described in detail below with reference to the embodiments shown in FIGS. 1 to 11.

In some embodiments, when the delivery tube holder 300 is in the first position, the atomizer 1000 is in the pre-cleaning state, as shown in FIG. 4A. When the delivery tube holder 300 is in the second position, the atomizer 1000 is in the cleaned state and the pre-triggering state, as shown in FIG. 5A. When the delivery tube holder 300 is in the third position, the atomizer 1000 is in the triggered state or the initial state, as shown in FIG. 6A. Thus, the atomizer 1000 is guided by the guide mechanism 440 to perform two spraying operations, that is, the pre-spraying and the formal drug-delivery spraying, within one stroke cycle.

In some embodiments, as shown in FIGS. 4D, 5D and 6D, the guide mechanism 440 may include: a first guide structure 330 arranged on an inner wall of the delivery tube holder 300, with a surface of the first guide structure 330 facing the mouthpiece 400 forming a first guide surface 331; and a second guide structure 430 arranged on an inner wall of the mouthpiece 400 (specifically, a mouthpiece body 410), with a surface of the second guide structure 430 facing the delivery tube holder 300 forming a second guide surface 431. Thus, during the pivoting of the delivery tube holder 300, the delivery tube holder 300 can move in the axial direction under the interaction of the first guide surface 331 and the second guide surface 431 that are in contact with each other, that is, can perform a composite motion of rotation and axial translation, so as to change the position of the delivery tube holder 300 in the axial direction L.

In some embodiments, during the pivoting of the delivery tube holder 300, the delivery tube holder 300 may move downward while rotating under the action of the first guide surface 331 and the second guide surface 431, and during the upward movement of the delivery tube holder 300, the delivery tube holder 300 may perform a simple upward translation without any rotation. The disclosure is not limited thereto.

In some embodiments, as shown in FIGS. 4D, 5D and 6D, when a first portion of the first guide surface 331 abuts on a second portion of the second guide surface 431, the delivery tube holder 300 is in the first position; when a third portion of the first guide surface 331 abuts on a fourth portion of the second guide surface 431, the delivery tube holder 300 is in the second position; and when the first guide surface 331 is separated from the second guide surface 431, the delivery tube holder 300 is in the third position. That is, the delivery tube holder 300 may be guided by the first guide surface 331 and the second guide surface 431 to be in the first position, is stopped by the first guide surface 331 and the second guide surface 431 to be in the second position, and then, after the first guide surface 331 is separated from the second guide surface 431 (i.e., having no guiding effect), enters the third position under the action of, for example, an upward thrust of a bottom spring 600 of the delivery tube holder 300. Thus, the delivery tube holder 300 can move from the first position to the second position and then to the third position in the axial direction L by means of the guide mechanism 440. Alternatively, it may also be configured that when a fifth portion of the first guide surface 331 abuts on a sixth portion of the second guide surface 431, the delivery tube holder 300 is in the third position. The disclosure is not limited thereto.

In some embodiments, as shown in FIGS. 4D, 5D, 6D, 7A and 7B, the first guide surface 331 of the first guide structure 330 on the delivery tube holder 300 may include: a guide portion 333 configured to guide the delivery tube holder 300 to move from the third position to the first position; and a stop portion 332 configured to guide the delivery tube holder 300 to move from the first position to the second position and stop the delivery tube holder 300 from moving from the second position to the first position. Thus, the guide mechanism 440 can guide the delivery tube holder 300 to move from top to bottom in the axial direction L, and stop the delivery tube holder 300 from moving from bottom to top in the axial direction L, such that the pre-spraying is performed before the formal spraying.

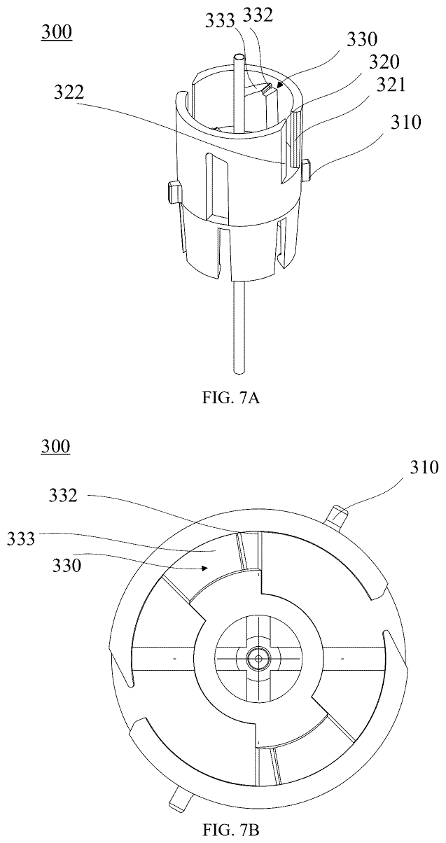

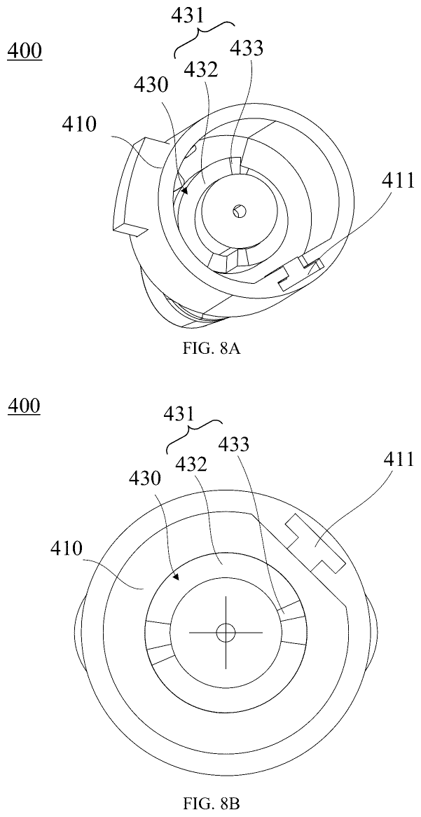

In some embodiments, as shown in FIGS. 7A and 7B, the guide portion 333 is an inclined surface, the stop portion 332 is a step surface. The stop portion 332 may include an axial flat portion 332-2 parallel to the axial direction L and a radial flat portion 332-1 parallel to a radial direction J of the delivery tube holder 300. The inclined surface may be a spiral surface, for example, a spiral surface that spirally extends upward from an inner bottom wall of the delivery tube holder 300 around a central axis of the delivery tube holder 300. Alternatively, the guide portion 333 may also be an inclined surface or a curved surface that extends in any direction and has a height gradually increasing from the inner bottom wall of the delivery tube holder 300. The disclosure is not limited to the example described above. Alternatively, the step surface may also include a flat portion or a curved portion extending in a direction at an angle relative to the axial direction L, and a flat portion or a curved portion extending in a direction at an angle relative to the radial direction J of the delivery tube holder 300. The disclosure is not limited to the example described above. Thus, the guide mechanism 440 can guide the delivery tube holder 300 to move from top to bottom in the axial direction L, and stop the delivery tube holder 300 from moving from bottom to top in the axial direction L, such that the pre-spraying is performed before the formal drug-delivery spraying.

In some embodiments, as shown in FIGS. 8A and 8B, the second guide surface 431 of the second guide structure 430 on the mouthpiece 400 may include an inclined portion 432 and a flat portion 433. The inclined portion 432 may be a spiral surface, for example, a spiral surface extending upward from an inner bottom wall of the mouthpiece 400 around a central axis of the mouthpiece 400. Alternatively, the guide portion 333 may also be an inclined surface or a curved surface that extends in any direction and has a height gradually increasing from the inner bottom wall of the mouthpiece 400. The disclosure is not limited to the example described above. The flat portion may be in the highest position of the second guide surface 431 in the axial direction L.

In some other embodiments, the second guide surface 431 of the second guide structure 430 on the mouthpiece 400 may include a guide portion configured to guide the delivery tube holder 300 to move from the third position to the first position and a stop portion configured to guide the delivery tube holder 300 to move from the first position to the second position. Correspondingly, the first guide surface 331 of the first guide structure 330 on the delivery tube holder 300 may only include an inclined portion and a flat portion.

In some embodiments, as shown in FIGS. 4D and 5D, the first portion is the highest point of the guide portion 333 of the first guide surface 331 in the axial direction L, and the second portion is the highest point of the second guide surface 431 in the axial direction L. The third portion is the radial flat portion 332-1 of the stop portion 332, and the fourth portion is the highest point of the second guide surface 431 in the axial direction L. Allowing the highest point of the first guide surface 331 in the axial direction L to come into contact with the highest point of the second guide surface 431 in the axial direction L enables the delivery tube holder 300 to be in the first position, that is, in the lowest position in the axial direction L. In addition, after the highest point of the first guide surface 331 is separated from the highest point of the second guide surface 431, the radial flat portion 332-1 of the stop portion 332 of the first guide surface 331 stops the highest point of the second guide surface 431, so as to temporarily stop the axial upward movement of the delivery tube holder 300 at the second position to enable the atomizer 1000 to perform pre-spraying before the formal spraying. In the embodiment described above, the second portion is the same as the fourth portion, both being the highest point of the second guide surface 431 in the axial direction L. In some other embodiments, the second portion and the fourth portion may be different portions. For example, the second portion is the highest point of the second guide surface 431 in the axial direction L, and the fourth portion is the second highest point of the second guide surface 431 in the axial direction L.

In some embodiments, as shown in FIGS. 4D, 5D and 6D, the first guide surface 331 further includes a transition portion 334. The transition portion 334 is located between the guide portion 333 and the stop portion 332 and is configured as a cambered surface. This facilitates the highest point of the second guide surface 431 to slide from the highest point of the first guide surface 331 to come into contact with the step surface of the first guide surface 331.

In some embodiments, the width of the first guide surface 331 in the radial direction J of the delivery tube holder 300 is different from the width of the second guide surface 431 in the radial direction J. Specifically, the width of the first guide surface 331 in the radial direction J may be greater than or less than the width of the second guide surface 431 in the radial direction J. This can reduce the frictional force when the first guide surface 331 and the second guide surface 431 slide relatively, such that the user's operation is more labor-saving.

In some embodiments, as shown in FIGS. 7A, 7B, 8A and 8B, the first guide structure 330 on the inner wall of the delivery tube holder 300 may extend along an inner circumferential wall of the delivery tube holder 300, and correspondingly, the second guide structure 430 on the inner wall of the mouthpiece 400 may extend around the central axis of the mouthpiece 400.

In some embodiments, as shown in FIGS. 7A, 7B, 8A and 8B, two or more first guide structures 330 may be provided on the inner wall of the delivery tube holder 300, and correspondingly, two or more second guide structures 430 may be provided on the inner wall of the mouthpiece 400. The number of the first guide structures 330 may be the same as or different from the number of the second guide structures 430. Thus, the atomizer 1000 can perform two or more stroke cycles of spraying when the delivery tube holder 300 rotates one circle, each stroke cycle of spraying including pre-spraying and formal drug-delivery spraying.

In some embodiments, the first guide structure 330 is integrally formed with the delivery tube holder 300, and the second guide structure 430 is integrally formed with the mouthpiece 400. This makes it easier to manufacture the first guide structure 330 and the second guide structure 430.

In some embodiments, as shown in FIGS. 4B, 5B, 6B and 10, the atomizer 1000 may further include an actuating member 500. The actuating member 500 can be actuated to drive the delivery tube holder 300 to pivot relative to the mouthpiece 400, and the pivoted delivery tube holder 300 can be guided by the guide mechanism 440 to move in the axial direction L.

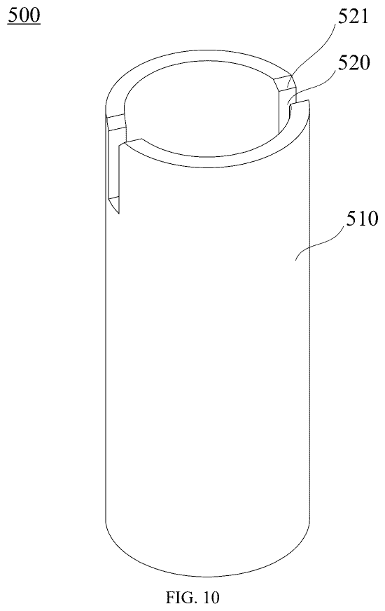

In some embodiments, as shown in FIG. 10, the actuating member 500 may have a cylindrical structure, and may be sleeved on the delivery tube holder 300 so as to drive the delivery tube holder 300 to rotate. Alternatively, the actuating member 500 may also be in other shapes, such as an oval shape. In addition, the actuating member 500 may be configured as the housing of the atomizer 1000, or the actuating member 500 may be connected to a housing 100 of the atomizer 1000, such that the delivery tube holder 300 can rotate or move in a spiral manner as the housing 100 rotates. The actuating member 500 may include an actuating member body 510.

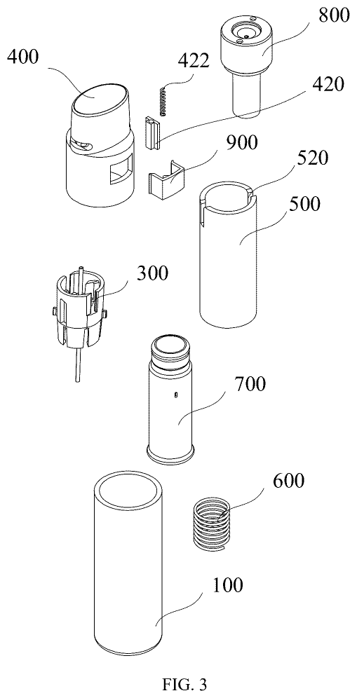

In some embodiments, as shown in FIG. 3, the mouthpiece 400 may be sleeved on the nozzle assembly 800 and/or the delivery tube holder 300, such that an atomized medicinal liquid is sprayed through one end of the mouthpiece 400. The mouthpiece 400 may include a mouthpiece body 410.

In some embodiments, as shown in FIGS. 4B, 5B, 6B and 9, the atomizer 1000 may further include a locking mechanism 530. The locking mechanism 530 switches the actuating member 500 between a locked position, in which the mouthpiece 400 stops the actuating member 500 from pivoting, and an unlocked position, in which the actuating member 500 can pivot relative to the mouthpiece 400.

In some embodiments, the locking mechanism 530 may include a first locking structure 420 arranged on the mouthpiece body 410 and a second locking structure 520 arranged on the actuating member body 510. As shown in FIGS. 4B, 5B and 6B, the first locking structure 420 may include a locking slider 421, and correspondingly, the second locking structure 520 may include a slot 521 for accommodating the locking slider 421. In this case, in the locked position, the locking slider 421 may be inserted into the slot 521 to stop the actuating member 500 from pivoting relative to the mouthpiece 400, as shown in FIG. 5B. In the implementation described above, the actuating member 500 can be effectively locked by means of a fixing member (i.e., the mouthpiece), which improves the locking reliability of the actuating member and thus avoids automatic trigger of the atomizer 1000 as much as possible. In some other embodiments, the first locking structure 420 may include a slot, and correspondingly, the second locking structure 520 may include a locking slider. Alternatively, the first locking structure 420 and the second locking structure 520 may also have other structures that can be selectively engaged and separated, such as a snap-fit structure, so as to lock or unlock the actuating member 500 relative to the mouthpiece 400.

In some embodiments, as shown in FIGS. 4B, 5B and 6B, the locking slider 421 can move relative to the mouthpiece body 410 in the axial direction L of the mouthpiece 400, and the slot 521 extends in the axial direction L from an end surface of the actuating member body 510 facing the mouthpiece 400. By means of providing the locking slider 421 that can move axially and the slot 521 that extends in the axial direction L as described above, the locking slider 421 can slide into the slot 521 by its own gravity when facing the slot 521, so as to lock the actuating member 500. Alternatively, the locking slider may also move in the radial direction J relative to the mouthpiece body 410, and correspondingly, the slot extends from a circumferential surface of the actuating member body 510 in the radial direction J. The disclosure is not limited to the example described above.

In some embodiments, in the locked position, a portion of the locking slider 421 in the axial direction L is inserted into the slot 521 to stop the actuating member 500 from pivoting relative to the mouthpiece 400; and in the unlocked position, the locking slider 421 abuts on the end surface of the actuating member body 510 facing the mouthpiece 400. Specifically, in the unlocked position, the locking slider 421 on the mouthpiece body 410 abuts on the end surface of the actuating member body 510 facing the mouthpiece 400. In this case, during the pivoting of the actuating member 500 relative to the mouthpiece 400, the locking slider 421 may slide on the end surface of the actuating member body 510 facing the mouthpiece 400 until the locking slider 421 is about to directly face the slot 521 in the actuating member body 510, as shown in FIG. 4B. Then, the actuating member 500 continues to be pivoted to allow the locking slider 421 to directly face the slot 521, and at this time, a portion of the locking slider 421 slides into the slot 521 of the actuating member body 510, such that the mouthpiece 400 and the actuating member 500 stop each other, as shown in FIG. 5B.

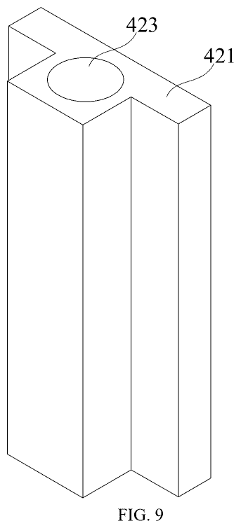

In some embodiments, as shown in FIG. 9, the locking slider 421 may have a T-shaped cross-section. The disclosure is not limited thereto. In addition, a hole 423 may be provided in the locking slider 421 for accommodating an elastic member 422 (described in detail below).

In some embodiments, as shown in FIG. 10, a side wall of the slot 521 may be provided with an inclined surface that is inclined from the side wall toward the end surface of the actuating member body 510 facing the mouthpiece 400. This makes it easy for the locking slider 421 to slide into the slot 521 when facing the slot 521, and also makes it easy for the locking slider 421 to return to and abut on the end surface of the actuating member body 510 facing the mouthpiece 400 after the locking slider 421 returns to the mouthpiece body 410.

In some embodiments, as shown in FIGS. 3, 8A and 8B, the mouthpiece body 410 may include a recess 411 for accommodating the locking slider 421, and the first locking structure 420 further includes the elastic member 422. One end of the elastic member 422 is fixed to an inner wall of the recess 411, and the other end of the elastic member 422 is fixed to the locking slider 421. The locking slider 421 may slide in the recess 411 of the mouthpiece body 410. When the actuating member 500 is in the locked position, a portion of the locking slider 421 may remain in the recess 411 of the mouthpiece body 410, and the other portion of the locking slider is inserted into the slot 521 of the actuating member body 510, such that the mouthpiece 400 and the actuating member 500 stop each other. In addition, providing the elastic member may further promote the movement of the locking slider 421. In some examples, the elastic member may be a spring, etc. Alternatively, the first locking structure 420 may include no elastic member. In this case, when the locking slider 421 faces the slot 521, the locking slider 421 may slide into the slot 521 only by its own gravity. In some other embodiments, the first locking structure 420 may further include other drive devices, such as an electric motor, to control the axial movement of the locking slider 421.

In some embodiments, when the actuating member 500 is in the unlocked position, the elastic member 422 is in a compressed state; and when the actuating member 500 is switched from the unlocked position to the locked position, the elastic member 422 returns to enable the locking slider 421 to enter the slot 521. The elastic force of the elastic member 422 may promote the locking slider 421 to be inserted into the slot 521 quickly and accurately, thereby improving the locking reliability of the actuating member 500.

In some embodiments, as shown in FIGS. 5B, 7A and 7B, a protrusion 310 is provided on the circumferential surface of the delivery tube holder 300. The protrusion 310 extends into the slot 521. In the locked position, the locking slider 421 abuts on the protrusion 310, such that when the delivery tube holder 300 moves toward the mouthpiece, the protrusion 310 pushes the locking slider 421 to return to the mouthpiece body 410. Specifically, as shown in FIG. 5B, in the locked position, the locking slider 421 is inserted into the slot 521 of the actuating member body 510 and abuts on the protrusion 310 of the delivery tube holder 300, so as to lock the actuating member 500. Then, for example, a switch button 900 can be pressed to enable the delivery tube holder 300 to continue to rotate, such that the delivery tube holder 300 can move toward the mouthpiece 400 under the action of the bottom spring 600 and drive, via the protrusion 310, the locking slider 421 to return into the mouthpiece body, as shown in FIG. 6B. At this time, the actuating member 500 returns to the unlocked position, the actuating member 500 continues to rotate to enable the locking slider 421 to abut on the end surface of the actuating member body 510 facing the mouthpiece again, and thus the atomizer 1000 returns to the initial state and is ready for the next spraying operation (dosing cycle).

In some embodiments, as shown in FIG. 4B, in the unlocked position, the protrusion 310 comes into contact with the slot 521, such that the actuating member 500 can push the delivery tube holder 300 to pivot via the protrusion 310. According to the implementation described above, the protrusion 310 on the delivery tube holder 300 not only enables the locking slider 421 to return to the mouthpiece, but also serves as a transmission member to transmit the rotational motion of the actuating member 500 to the delivery tube holder 300, thereby simplifying the structure of the atomizer 1000 to make the atomizer 1000 more compact and reduce the cost. In some other embodiments, the actuating member may drive the delivery tube holder 300 to rotate by means of a transmission member having other structures, for example, a protrusion provided on the actuating member body 510.

In some embodiments, when the delivery tube holder 300 is in the first position and the third position, the actuating member 500 is in the unlocked position, as shown in FIGS. 4A, 4B, 6A and 6B; and when the delivery tube holder 300 is in the second position, the actuating member 500 is in the locked position, as shown in FIGS. 5A and 5B. This can prevent the atomizer 1000 from being automatically triggered, after entering the cleaned state (i.e., the pre-triggering state), due to the user continuing to rotate the actuating member 500. Specifically, when the delivery tube holder 300 is in the first position, the locking slider 421 abuts against the end surface of the actuating member 500 facing the mouthpiece 400, such that the actuating member 400 is in the unlocked position, as shown in FIGS. 4A and 4B. When the delivery tube holder 300 is in the second position, a portion of the locking slider 421 is inserted into the slot 521 and abuts on the protrusion 310, such that the actuating member 500 is in the locked position, as shown in FIGS. 5A and 5B. When the delivery tube holder 300 is in the third position, the locking slider is pushed by the protrusion 310 to return to the mouthpiece 400, such that the actuating member 500 is in the unlocked position, as shown in FIGS. 6A and 6B.

In some embodiments, as shown in FIGS. 5C, 6C and 11, the atomizer 1000 may further include a switch button 900. In the locked position, the switch button 900 can be pressed to drive the delivery tube holder 300 to pivot to separate the first guide surface 331 from the second guide surface 431, so as to enable the delivery tube holder 300 to move from the second position to the third position under the action of the bottom spring 600, such that the atomizer 1000 enters the triggered state from the pre-triggering state (i.e., the cleaned state).

In some embodiments, as shown in FIGS. 4C, 5C, 6C and 11, a first inclined surface 911 is provided at an end portion 910 of the switch button 900 facing the delivery tube holder 300, the first inclined surface 911 is configured to be inclined from the end 910 away from the delivery tube holder 300, an opening 320 is provided in a circumferential wall of the delivery tube holder 300, and the opening 320 is configured to accommodate the end portion 910. Thus, when the switch button 900 is pressed, the first inclined surface 911 on the switch button 900 can push a side wall on the opening 320 of the delivery tube holder 300 so as to push the delivery tube holder 300 to pivot.

In some embodiments, as shown in FIGS. 5C, 6C, 7B and 11, a third inclined surface 912 opposite to the first inclined surface 911 is further provided at the end portion of the switch button 900. The third inclined surface 912 is configured to be inclined from the end portion 910 away from the delivery tube holder 300, which can reduce resistance to make it easy for a second inclined surface 323 (described in detail below) of the delivery tube holder 300 to come into contact with the end portion 910 of the switch button 900.

In some embodiments, as shown in FIG. 4C, in the unlocked position, the end portion 910 faces the circumferential wall of the delivery tube holder 300. This can ensure that the switch button 900 cannot be pressed when the atomizer 1000 does not enter the pre-triggering state, thereby ensuring normal drug delivery of the atomizer 1000.

In some embodiments, as shown in FIG. 5C, in the locked position, the end portion 910 faces a first side wall 321 of the opening 320, and when the switch button 900 is pressed, the first inclined surface 911 can come into contact with the first side wall 321 of the opening 320 to push the delivery tube holder 300 to pivot. With the arrangement in which the end portion 910 of the switch button 900 faces the first side wall 321 of the opening 320 of the delivery tube holder 300 when in the locked position, when the switch button 900 is pressed, the end portion 910 of the switch button 900 can enter the opening 320 to enable the first inclined surface 911 to come into contact with the first side wall 321 of the opening 320. In this way, the switch button 900 is used to drive the delivery tube holder 300 to rotate to enable the atomizer 1000 to enter the triggered state from the pre-triggering state.

In some embodiments, as shown in FIGS. 4C, 5C, 6C and 7A, the second inclined surface 323 inclined from a second side wall 322 toward the switch button 900 is provided on the second side wall 322 of the opening 320 facing the first side wall 321; and as shown in FIG. 6C, when the atomizer 1000 is in the triggered state, the actuating member 500 continues to be pivoted to cause the second inclined surface 323 to come into contact with the end portion 910, so as to enable the switch button 900 to move away from the delivery tube holder 300. Specifically, after the switch button 900 is pressed to enable the atomizer 1000 to enter the triggered state, the actuating member 500 returns to the unlocked position, and at this time, the actuating member 500 may be rotated to drive the delivery tube holder 300 to rotate, so as to cause the second inclined surface 323 of the opening 320 of the delivery tube holder 300 to come into contact with the end portion 910 of the switch button 900. In this way, the rotation of the delivery tube holder 300 can push the switch button 900 to return to the initial state to be ready for the next spraying operation.

To this end, the actuating member 500 can drive the delivery tube holder 300 to rotate in the unlocked position to switch the atomizer 1000 from the initial state to the pre-cleaning state and from the pre-cleaning state to the cleaned state and the pre-triggering state, and the switch button 900 can be pressed in the locked position to push the delivery tube holder 300 to continue to rotate to enable the atomizer 1000 to enter the triggered state from the pre-triggering state. After the atomizer 1000 enters the triggered state, the actuating member 500 also returns to the unlocked position. At this time, the actuating member 500 can be rotated to drive the delivery tube holder 300 to rotate, so as to enable the atomizer 1000 to return to the initial state.

In some embodiments, the atomizer 1000 may further include a bottom spring 600 that is arranged at the bottom of the delivery tube holder 300 and configured to apply a thrust to the delivery tube holder 300 to drive the delivery tube holder 300 to move toward the mouthpiece 400. Specifically, when the first guide surface 331 is separated from the second guide surface 431, the delivery tube holder 300 can move from the second position to the third position under the action of the thrust of the bottom spring 600. In this way, the delivery tube holder 300 moves from the first position to the second position and the third position under the combined action of the guide mechanism 440 and the bottom spring 600, so as to enable the atomizer 1000 to perform the pre-spraying and the formal drug-delivery spraying.

In some embodiments, the atomizer 1000 may further include other members, such as a housing 100, a container 700 for holding the liquid to be atomized, a spraying assembly 800 for atomizing the liquid and spraying the atomized liquid, and a dust cover 200.

According to some embodiments of the disclosure described above, the atomizer 1000 may be, but does not necessarily, operated as follows.

When the atomizer 1000 is in the initial state, the first guide structure 330 on the inner wall of the delivery tube holder 300 is separated from the second guide structure 430 on the inner wall of the mouthpiece 400, the locking slider 421 on the mouthpiece 400 abuts on the end surface of the actuating member body 510 facing the mouthpiece 400, and the end portion 910 of the switch button 900 faces the circumferential wall of the delivery tube holder 300.

From the initial state, the user holds the housing 100 and rotates the housing 100 by an angle, and the actuating member 500 drives the delivery tube holder 300 to rotate along with the housing 100, such that the first guide structure 330 on the delivery tube holder 300 comes into contact with the second guide structure 430 on the mouthpiece 400. At this time, the delivery tube holder 300 may move downward in a spiral manner under the action of the first guide structure 330 and the second guide structure 430. As the actuating member 500 rotates, the slot 521 in the actuating member 500 approaches the locking slider 421 on the mouthpiece 400, as shown in FIG. 4B, and the end portion 910 of the switch button 900 also approaches the first side wall 321 of the opening 320 of the delivery tube holder 300, as shown in FIG. 4C. The actuating member 500 continues to rotate until the highest point of the first guide structure 330 comes into contact with the highest point of the second guide structure 430, and at this time, the delivery tube holder 300 is in the first position, that is, the atomizer 1000 enters the pre-cleaning state, as shown in FIG. 4A.

When the atomizer 1000 is in the pre-cleaning state, the bottom spring 600 of the delivery tube holder 300 is compressed to the maximum extent, as shown in FIG. 4A. At this time, the actuating member 500 continues to rotate, the highest point of the first guide structure 330 is separated from the highest point of the second guide structure 430, the delivery tube holder 300 moves toward the mouthpiece 400 in the axial direction L under the action of the upward thrust of the bottom spring 600 until the step surface (specifically, the radial flat portion) of the first guide structure 330 comes into contact with the highest point of the second guide structure 430, and at this time, the delivery tube holder 300 is in the second position, that is, the atomizer 1000 enters the cleaned state, as shown in FIG. 5A. The process described above is a pre-spraying process of the atomizer.

When the atomizer 1000 is in the cleaned state (also referred to as a pre-triggering state), a portion of the locking slider 421 on the mouthpiece 400 is inserted into the slot 521 of the actuating member body 510 and abuts on the protrusion 310 on the circumferential wall of the delivery tube holder 300, such that the actuating member 500 is in the locked position, as shown in FIG. 5B. In addition, the end portion 910 of the switch button 900 faces the first side wall 321 of the delivery tube holder 300, as shown in FIG. 5C. In the locked position, if the user does not apply an external force by means of other components, the atomizer 1000 does not perform an atomization or spraying operation, even if the atomizer 1000 is subjected to an external force, such as impact or shaking, or even accidents such as slipping from a height. Thus, the atomizer 1000 is effectively locked in this state.

When the user wants to use the atomizer 1000 to perform formal drug-delivery spraying, the user may apply a radial inward thrust to the switch button 900, that is, press the switch button 900. The switch button 900 is pressed such that the first inclined surface 911 on the switch button 900 comes into contact with the first side wall 321 of the opening 320 of the delivery tube holder 300 and pushes the first side wall 321 to move, so as to drive the delivery tube holder 300 to further rotate. At this time, the protrusion 310 on the delivery tube holder 300 also moves a distance in the slot 521 along with the delivery tube holder 300. When the second guide surface 431 of the second guide structure 430 of the mouthpiece 400 is separated from the first guide surface 331 of the first guide structure 330 of the delivery tube holder 300, the delivery tube holder 300 moves toward the mouthpiece 400 in the axial direction L under the action of the upward thrust of the bottom spring 600 to enable the atomizer 1000 to enter the triggered state, as shown in FIG. 6A. In addition, the protrusion 310 on the delivery tube holder 300 pushes the locking slider 421 to return into the mouthpiece body, so as to enable the actuating member 500 to return to the unlocked position, as shown in FIG. 6B.

Then, the user continues to rotate the housing 100 by an angle to enable the actuating member 500 to continue to drive the delivery tube holder 300 to rotate along with the housing 100. The locking slider 421 returns to and abuts on the end surface of the actuating member body 510 facing the mouthpiece 400. In addition, as shown in FIG. 6C, the second inclined surface 323 of the second side wall 322 of the delivery tube holder 300 comes into contact with the end portion 910 of the switch button 900 and pushes the switch button 900 to return to the original position, such that the atomizer 1000 returns to the initial position. At this point, the atomizer 1000 completes one stroke cycle of spraying.

An atomizer 1000′ according to some specific embodiments of the disclosure in which no pre-spraying is performed will be described in detail below with reference to the embodiments shown in FIGS. 12A to 12C. It should be noted herein that, except the feature that no limiting step for pre-spraying is provided on a first guide surface of a first guide structure 330′ in the embodiments in which no pre-spraying is performed, other features of the delivery tube holder of the atomizer 1000′ in the embodiments in which no pre-spraying is performed and the features of other components such as a nozzle, an actuating member, a locking mechanism and a switch button are the same as the features of the delivery tube holder of the atomizer 1000 in the embodiments in which pre-spraying is performed and the features of the components such as the nozzle, the actuating member, the locking mechanism and the switch button, and will not be described in detail herein for the sake of brevity. Only the differences between the atomizer 1000′ in the embodiments in which no pre-spraying is performed and the atomizer 1000 in the embodiments in which pre-spraying is performed will be described in detail below.

In some embodiments, when the delivery tube holder 300 is in the first position, the atomizer 1000′ is in a pre-triggering state, as shown in FIG. 12A. When the delivery tube holder 300 is in the third position, the atomizer 1000′ is in a triggered state or an initial state, as shown in FIG. 13A. Thus, the atomizer 1000′ is guided by the guide mechanism 440′ to perform formal drug-delivery spraying and to be ready for the next drug-delivery spraying within one stroke cycle. In some embodiments, the guide mechanism 440′ may include: a first guide structure 330′ arranged on an inner wall of the delivery tube holder 300, with a surface of the first guide structure 330′ facing the mouthpiece 400 forming the first guide surface; and a second guide structure 430 arranged on an inner wall of the mouthpiece 400, with a surface of the second guide structure 430 facing the delivery tube holder 300 forming a second guide surface. Thus, during the pivoting of the delivery tube holder 300, the delivery tube holder 300 can move in the axial direction under the interaction of the first guide surface and the second guide surface that are in contact with each other, that is, can perform a composite motion of rotation and axial translation, so as to change the position of the delivery tube holder 300 in the axial direction L.

In some embodiments, during the pivoting of the delivery tube holder 300, the delivery tube holder 300 may move downward while rotating under the action of the first guide surface and the second guide surface, and during the upward movement of the delivery tube holder 300, the delivery tube holder 300 may perform a simple upward translation without any rotation. The disclosure is not limited thereto. When the delivery tube holder is in the first position, the highest point of the first guide surface in the axial direction comes into contact with the highest point of the second guide surface in the axial direction, as shown in FIG. 12A, and at this time, the delivery tube holder is farthest from the mouthpiece. Then, after the first guide surface is separated from the second guide surface, the delivery tube holder moves to the third position.

In some embodiments, the first guide surface may include an inclined portion and a flat portion. The inclined portion may be a spiral surface, for example, a spiral surface that spirally extends upward from an inner bottom wall of the delivery tube holder 300 around a central axis of the delivery tube holder 300. Alternatively, the inclined portion may also be an inclined surface or a curved surface that extends in any direction and has a height gradually increasing from the inner bottom wall of the delivery tube holder 300. The disclosure is not limited to the example described above. The flat portion may be in the highest position of the first guide surface in the axial direction L.

In some embodiments, the second guide surface 431 may also include an inclined portion and a flat portion. The inclined portion may be a spiral surface, for example, a spiral surface extending upward from an inner bottom wall of the mouthpiece 400 around a central axis of the mouthpiece 400. Alternatively, the inclined portion may also be an inclined surface or a curved surface that extends in any direction and has a height gradually increasing from the inner bottom wall of the mouthpiece 400. The disclosure is not limited to the example described above. The flat portion may be in the highest position of the second guide surface in the axial direction L.

In some embodiments, when the delivery tube holder 300 is in the first position, the actuating member 500 is in an unlocked position (as shown in FIGS. 12A and 12B); and when the delivery tube holder 300 is in the third position, the actuating member 500 is in a locked position, as shown in FIGS. 13A and 13B. When the delivery tube holder 300 is in the first position, a locking slider 421 may be inserted into a slot 521 to stop the actuating member 500 from pivoting relative to the mouthpiece 400, so as to enable the actuating member to be in the locked position, as shown in FIG. 12B. In the implementation described above, the actuating member 500 can be effectively locked by means of a fixing member (i.e., the mouthpiece), which improves the locking reliability of the actuating member and thus avoids automatic trigger of the atomizer 1000′ as much as possible. The locking slider 421 can move relative to the mouthpiece body in the axial direction L of the mouthpiece 400, and the slot 521 extends in the axial direction L from an end surface of the actuating member body 510 facing the mouthpiece 400. By means of providing the locking slider 421 that can move axially and the slot 521 that extends in the axial direction L as described above, the locking slider 421 can slide into the slot 521 by its own gravity when facing the slot 521, so as to lock the actuating member 500.

In addition, when the delivery tube holder 300 is in the first position, a portion of the locking slider 421 is inserted into the slot 521 and abuts on a protrusion 310, such that the actuating member 500 is in the locked position, as shown in FIGS. 12A and 12B. When the delivery tube holder 300 is in the third position, the locking slider is pushed by the protrusion 310 to return to the mouthpiece 400, such that the actuating member 500 is in the unlocked position, as shown in FIGS. 13A and 13B.

In some embodiments, as shown in FIG. 12C, in the locked position, the switch button 900 can be pressed to drive the delivery tube holder 300 to pivot to separate the first guide surface from the second guide surface, so as to enable the delivery tube holder 300 to move from the first position to the third position under the action of a bottom spring 600, such that the atomizer 1000′ enters the triggered state from the pre-triggering state. As shown in FIG. 13C, when the atomizer 1000′ is in the triggered state, the actuating member 500 continues to be pivoted to cause a second inclined surface 323 to come into contact with an end portion 910, so as to enable the switch button 900 to move away from the delivery tube holder 300. Specifically, after the switch button 900 is pressed to enable the atomizer 1000′ to enter the triggered state, the actuating member 500 returns to the unlocked position, and at this time, the actuating member 500 may be rotated to drive the delivery tube holder 300 to rotate, so as to cause the second inclined surface 323 of the opening 320 of the delivery tube holder 300 to come into contact with the end portion 910 of the switch button 900. In this way, the rotation of the delivery tube holder 300 can push the switch button 900 to return to the initial state to be ready for the next spraying operation.

According to some embodiments of the disclosure described above, the atomizer 1000′ may be, but does not necessarily, operated as follows.

When the atomizer 1000′ is in the initial state, the first guide structure 330′ on the inner wall of the delivery tube holder 300 is separated from the second guide structure 430 on the inner wall of the mouthpiece 400, the locking slider 421 on the mouthpiece 400 abuts on the end surface of the actuating member body 510 facing the mouthpiece 400, and the end portion 910 of the switch button 900 faces the circumferential wall of the delivery tube holder 300.

From the initial state, the user holds the housing and rotates the housing by an angle, and the actuating member 500 drives the delivery tube holder 300 to rotate along with the housing, such that the first guide structure 330′ on the delivery tube holder 300 comes into contact with the second guide structure 430 on the mouthpiece 400. At this time, the delivery tube holder 300 may move downward in a spiral manner under the action of the first guide structure 330′ and the second guide structure 430. As the actuating member 500 rotates, the slot 521 in the actuating member 500 approaches the locking slider 421 on the mouthpiece 400, and the end portion 910 of the switch button 900 also approaches the first side wall 321 of the opening 320 of the delivery tube holder 300, as shown in FIG. 12C. The actuating member 500 continues to rotate until the highest point of the first guide structure 330′ comes into contact with the highest point of the second guide structure 430, and at this time, the delivery tube holder 300 is in the first position, that is, the atomizer 1000 enters the pre-triggering state, as shown in FIG. 12A, and the locking slider 421 slides into the slot 521 to lock the actuating member 500, as shown in FIG. 12B.

When the atomizer 1000′ is in the pre-triggering state, the bottom spring 600 of the delivery tube holder 300 is compressed to the maximum extent, as shown in FIG. 12A. At this time, when the user wants to use the atomizer 1000′ to perform formal drug-delivery spraying, the user may apply a radial inward thrust to the switch button 900, that is, press the switch button 900. The switch button 900 is pressed such that the first inclined surface 911 on the switch button 900 comes into contact with the first side wall 321 of the opening 320 of the delivery tube holder 300 and pushes the first side wall 321 to move, so as to drive the delivery tube holder 300 to further rotate. At this time, the protrusion 310 on the delivery tube holder 300 also moves a distance in the slot 521 along with the delivery tube holder 300. When the second guide surface of the second guide structure 430 of the mouthpiece 400 is separated from the first guide surface of the first guide structure 330 of the delivery tube holder 300, the delivery tube holder 300 moves toward the mouthpiece 400 in the axial direction L under the action of the upward thrust of the bottom spring 600 to enable the atomizer 1000′ to enter the triggered state, as shown in FIG. 13A. In addition, the protrusion 310 on the delivery tube holder 300 pushes the locking slider 421 to return into the mouthpiece body, so as to enable the actuating member 500 to return to the unlocked position, as shown in FIG. 13B.

Then, the user continues to rotate the housing by an angle to enable the actuating member 500 to continue to drive the delivery tube holder 300 to rotate along with the housing. The locking slider 421 returns to and abuts on the end surface of the actuating member body 510 facing the mouthpiece 400. In addition, as shown in FIG. 13C, the second inclined surface 323 of the second side wall 322 of the delivery tube holder 300 comes into contact with the end portion 910 of the switch button 900 and pushes the switch button 900 to return to the original position, such that the atomizer 1000′ returns to the initial position. At this point, the atomizer 1000′ completes one stroke cycle of spraying.

What is claimed is:

1. An atomizer, comprising:
a mouthpiece, a first end of the mouthpiece being configured to be placed at a mouth of a user;
a delivery tube holder located at a second end of the mouthpiece opposite to the first end; and
a guide mechanism configured to guide the delivery tube holder to move between a first position and a third position in an axial direction of the mouthpiece,
wherein the delivery tube holder is closest to the mouthpiece in the axial direction when the delivery tube holder is in the third position, and the delivery tube holder is farthest from the mouthpiece in the axial direction when the delivery tube holder is in the first position; and
wherein the atomizer performs spraying during the movement of the delivery tube holder from the first position to the third position, and the atomizer performs liquid metering during the movement of the delivery tube holder from the third position to the first position.

2. The atomizer according to claim 1, wherein there is a second position between the first position and the third position, and the guide mechanism is configured to guide the delivery tube holder to move between the first position, the second position and the third position in the axial direction of the mouthpiece; and
wherein the atomizer performs liquid metering during the movement of the delivery tube holder from the third position to the first position, the atomizer performs a first spraying operation during the movement of the delivery tube holder from the first position to the second position, and the atomizer performs a second spraying operation during the movement of the delivery tube holder from the second position to the third position, so as to complete one dosing cycle.

3. The atomizer according to claim 2, wherein the guide mechanism comprises:
a first guide structure arranged on an inner wall of the delivery tube holder, with a surface of the first guide structure facing the mouthpiece forming a first guide surface; and
a second guide structure arranged on an inner wall of the mouthpiece, with a surface of the second guide structure facing the delivery tube holder forming a second guide surface.

4. The atomizer according to claim 3, wherein
when a first portion of the first guide surface abuts on a second portion of the second guide surface, the delivery tube holder is in the first position;
when a third portion of the first guide surface abuts on a fourth portion of the second guide surface, the delivery tube holder is in the second position; and
when the first guide surface is separated from the second guide surface, the delivery tube holder is in the third position.

5. The atomizer according to claim 4, wherein the first guide surface comprises:
a guide portion configured to guide the delivery tube holder to move from the third position to the first position; and
a stop portion configured to guide the delivery tube holder to move from the first position to the second position and stop the delivery tube holder from moving from the second position to the first position.

6. The atomizer according to claim 5, wherein the guide portion is an inclined surface, and the stop portion is a step surface and comprises an axial flat portion parallel to the axial direction and a radial flat portion parallel to a radial direction of the delivery tube holder.

7. The atomizer according to claim 6,
wherein the first portion is the highest point of the guide portion in the axial direction, and the second portion is the highest point of the second guide surface in the axial direction; and
wherein the third portion is the radial flat portion of the stop portion, and the fourth portion is the same portion as the second portion.

8. The atomizer according to claim 5, wherein the first guide surface further comprises a transition portion located between the guide portion and the stop portion and configured as a cambered surface.

9. The atomizer according to claim 3, wherein a width of the first guide surface in a radial direction of the delivery tube holder is different from a width of the second guide surface in the radial direction.

10. The atomizer according to claim 3, wherein the first guide structure is integrally formed with the delivery tube holder, and the second guide structure is integrally formed with the mouthpiece.

11. The atomizer according to claim 4, further comprising an actuating member, wherein the actuating member is capable of being actuated to drive the delivery tube holder to pivot relative to the mouthpiece, and the pivoted delivery tube holder is capable of being guided by the guide mechanism to move in the axial direction.

12. The atomizer according to claim 11, further comprising a locking mechanism, wherein the locking mechanism switches the actuating member between a locked position, in which the mouthpiece stops the actuating member from pivoting, and an unlocked position, in which the actuating member is capable of pivoting relative to the mouthpiece.

13. The atomizer according to claim 12, wherein
when the delivery tube holder is in the first position and the third position, the actuating member is in the unlocked position; and
when the delivery tube holder is in the second position, the actuating member is in the locked position.

14. The atomizer according to claim 12, further comprising a switch button, wherein in the locked position, the switch bottom is capable of being pressed to drive the delivery tube holder to pivot to separate the first guide surface from the second guide surface.

15. The atomizer according to claim 12,
further comprising a bottom spring arranged at the bottom of the delivery tube holder, wherein the bottom spring is configured to apply a thrust to the delivery tube holder to drive the delivery tube holder to move toward the mouthpiece, and
when the first guide surface is separated from the second guide surface, the delivery tube holder is capable of moving from the second position to the third position under the action of the thrust of the bottom spring.

16. The atomizer according to claim 12, wherein the locking mechanism comprises:
a locking slider provided on the mouthpiece;
a slot provided in a circumferential wall of the actuating member; and
a protrusion provided on a circumferential surface of the delivery tube holder and extending into the slot,
wherein when the delivery tube holder is in the first position, the locking slider abuts against an end surface of the actuating member facing the mouthpiece, such that the actuating member is in the unlocked position;
wherein when the delivery tube holder is in the second position, a portion of the locking slider is inserted into the slot and abuts on the protrusion, such that the actuating member is in the locked position; and
wherein when the delivery tube holder is in the third position, the locking slider is pushed by the protrusion to return to the mouthpiece, such that the actuating member is in the unlocked position.

17. The atomizer according to claim 2, wherein
when the delivery tube holder is in the first position, the atomizer is in a pre-cleaning state;
when the delivery tube holder is in the second position, the atomizer is in a cleaned state and a pre-triggering state; and
when the delivery tube holder is in the third position, the atomizer is in a triggered state or an initial state.

18. The atomizer according to claim 12, wherein
when the delivery tube holder is in the first position, the actuating member is in the locked position; and
when the delivery tube holder is in the third position, the actuating member is in the unlocked position.

19. The atomizer according to claim 12, wherein the locking mechanism comprises:
a locking slider provided on the mouthpiece;
a slot provided in a circumferential wall of the actuating member; and
a protrusion provided on a circumferential surface of the delivery tube holder and extending into the slot,
wherein when the delivery tube holder is in the first position, a portion of the locking slider is inserted into the slot and abuts on the protrusion, such that the actuating member is in the locked position; and
wherein when the delivery tube holder is in the third position, the locking slider is pushed by the protrusion to return to the mouthpiece, such that the actuating member is in the unlocked position.
